(19) United States
(12) Patent Application Publication
(10) Pub. No.: US 2005/0209612 A1
(43) Pub. Date: Sep. 22, 2005

(54) Endoscopic suturing assembly and associated methodology using a temperature biased suture needle

(76) Inventor: Naomi L. Nakao, New York, NY (US)

Correspondence Address: WOOD, HERRON & EVANS, LLP, 2700 CAREW TOWER, 441 VINE STREET, CINCINNATI, OH, 45202, US

(21) Appl. No.: 11/069,683

(22) Filed: Mar. 1, 2005

Related U.S. Application Data
(60) Provisional application No. 60/549,275, filed on Mar. 2, 2004.

(51) Int. Cl. A61B 17/04

(52) U.S. Cl. 606/144

(57) ABSTRACT

An endoscopic suture needle and related surgical endoscopic suturing devices are to be used in conjunction with an endoscope. The invention relates to suturing of internal body tissue as part of a surgical procedure which may be diagnostic, therapeutic or both. In accordance with the present invention, there is provided an endoscopic surgery system comprising a temperature biased suture needle, a needle grasping device, and an elongated catheter or other delivery tube, a endoscopic surgery system configured for use in conjunction with a flexible or rigid endoscope insertion member.

This application claims the benefit of the priority of U.S. Provisional Application Ser. No. 60/549,275, filed on Mar. 2, 2004, entitled “Temperature Biased Suture Needle,” which application is incorporated herein by reference in its entirety.

### FIELD OF THE INVENTION

The present invention relates to a surgical instrument assembly for use in suturing inside internal body cavities of a patient, and more specifically to an instrument assembly for use in conjunction with a flexible or rigid endoscope to suture tissue within the body. This invention has particular applicability for suturing in conjunction with an endoscope inside internal body cavities of a patient, for example, inside the abdomen by gaining access through an existing orifice.

### BACKGROUND OF THE INVENTION

In a conventional abdominal surgical procedure, one or more incisions are created in the abdominal wall in order to enter the abdominal cavity. Surgical procedures to remove diseased tissue or organs are currently performed via open or laparoscopic surgery. In addition to major abdominal operations such as colon resection, gall bladder removal, and stomach resections, surgery for morbid obesity (bariatric surgery) is being performed with greater and greater frequency due to the increasing prevalence of morbid obesity and its complications.

The high incidence of obesity its related medical problems have reached epidemic proportions in the United States affecting more than 30% of the adult population and accounting for nearly 300,000 deaths annually. Bariatric procedures most commonly performed include vertical banded gastroplasty, gastric banding, and Roux-en-Y gastric bypass (RNYGB). Morbidity and mortality resulting from these operations is relatively high.

These complex and invasive surgical procedures require general anesthesia, surgical incisions, lengthy periods of time in the hospital, significant use of medication for management of postoperative pain and lengthy periods of convalescence. Surgical procedures to treat morbidly obese patients have a high incidence of complications and thus limit the number of patients who can benefit from these procedures. Surgery for morbid obesity is currently performed through a large abdominal incision. The operation entails exclusion of a large portion of stomach, and a bypass procedure of the small intestine. Oftentimes the patient has had prior surgery causing adhesions, which bind the intestines together. In that case the surgeon must first dissect these adhesions and free the bowel in order to reach the operative site.

While laparoscopic surgery, which is a less invasive procedure, has become the standard of surgical care for numerous disease processes, complications from laparoscopic bariatric surgery are comparable to those resulting from open procedures. The surgery is technically more difficult and takes two to three hours longer than the open operation. Consequently longer anesthesia time is required, increasing patient morbidity. In order to perform gastric bypass surgery laparoscopically, the abdomen requires distention with air, which impinges on the patient's lungs thereby decreasing breathing capacity.

Providing an option for surgery that may be performed through an existing orifice via the flexible endoscope would offer a less invasive approach. Because flexible endoscopic procedures are classically performed under conscious sedation and do not require an incision to enter the body, they are naturally less invasive. Consequently, morbidity and mortality would be reduced, convalescence time and hospital stay would be shortened, post-operative pain virtually eliminated and cost savings provided.

Yet, such procedures are currently limited to examinations that include biopsy and polypectomy within the lumen of the gastrointestinal tract. One of the significant reasons for this limitation is the lack of the ability, with current surgery assemblies and techniques, to perform suturing and/or stapling through the narrow working channel of the flexible endoscope.

Although there appear to be no commercial devices on the market that enable suturing through the working channel of the flexible endoscope, U.S. Pat. No. 5,037,433 to Wilk et al. describes an endoscopic suturing device that comprises an endoscope and a needle having a mechanical spring bias construction tending to bend the needle into an arcuate configuration. The needle is disposed in a straightened configuration while inside the endoscope. The surgical instrument further comprises an ejector device in the form of an elongate flexible rod member slidably disposed inside the inner tubular member proximally of the needle for ejecting a needle, which mechanically assumes an arcuate configuration subsequent to its ejection.

Based on the disclosure and drawings of the '433 patent, the mechanical spring biased or elastic tendency of the needle tends to bend a needle in an arcuate configuration. As such, this pre-stressed plastic or metal needle may be deformed (i.e. straightened) by mechanical stresses on the needle being confined in a generally straight biopsy channel of an endoscope, deforming the needle to render it generally straight. The mechanical stresses are provided and maintained by the walls of the biopsy channel into which the needle is inserted. Once the needle is ejected out of the biopsy channel by a rod, the stresses are removed, and the free needle immediately assumes its pre-stressed arcuate configuration under the direction of its normal elastic properties.

The device described in the '433 patent presents the various drawbacks and problems. First, the flexible endoscope is constructed in such a fashion as to allow only a 1 cm “stiff length” or less to pass through its biopsy or working channel. Any embodiment with a stiff length longer than 1 cm will not be capable of being passed through the working channel when the endoscope is bent, and will prevent the flexible endoscope from bending when housed inside its working channel. Consequently, only a device that is sufficiently malleable to bend relatively easily along with the endoscope may be passed through its working channel. Suturing requires a rigid needle shaped in an arcuate form. When such a needle is plunged into the target tissue in one location, it will exit the tissue at a second location in a predictable manner because of the needle's arcuate configuration and stiff or rigid state. Accordingly, there are two important requirements that a suture needle must fulfill if it were to be used through the working channel of a flexible endoscope. On one hand, it must be malleable enough to be passed through the working channel of a flexible endoscope while an endoscope is bent to its maximum capacity, while on the other hand it must assume a rigid arcuate state in readiness for the suturing operation upon ejection. If the spring biased needle described in the '433 patent were to be sufficiently malleable to be passed through the working channel of an endoscope, it would surely be too malleable to enter and exit tissue in a reliable fashion. If a needle were to be formed from a material stiff enough to effectively and consistently enter and exit tissue, it would surely not be malleable enough pass through the working channel of a flexible endoscope.

A further problem that the device described in the '433 patent presents is its lack of anticipation of the difficulty presented in grasping the suture needle with the manipulation device. Just as in open and laparoscopic surgery, a suture needle must be grasped firmly so as not to rotate on its axis during the plunging of a needle into tissue. If the needle is permitted to rotate on its own axis it will only push against the tissue but will not enter it. Grasping a needle with jaw-closure-force being transmitted through a short rigid shaft, as is done during open or laparoscopic surgery is significantly different from grasping a needle with closure force being transmitted through a long flexible shaft. The latter forces required to close the jaws tightly are infinitely greater than in the former case. The '433 patent does not address such an issue. No special construction of the needle's shaft to enhance grasping is described, and the description of the grasping device does not anticipate any of the abovementioned difficulty.

Lastly, the '433 patent does not address the attachment of the suturing thread to the needle. Spring biased metals do not behave as stainless steel does. In the case of the stainless steel suture needle, the suture thread is inserted into a cavity at the proximal end of the needle and the metal is crimped over the thread. In the case of a needle made of a spring biased metal, the metal is too soft to retain the thread by mere crimping.

Therefore, it would be desirable to address the shortcomings and drawbacks of the prior art and to specifically provide an instrument assembly for suturing in p laces internal to a patient's body utilizing flexible or rigid endoscopes inserted primarily, though not exclusively, through existing body orifices.

It is further desirable to provide such an instrument assembly for performing surgery through said endoscope, whereby an instrument assembly may be passed through the narrow, preferably flexible working channel of said endoscope.

It is also desirable to address suturing concerns with a needle that is malleable enough to go through the working channel of the endoscope without inhibiting said endoscope's bending maneuverability, and yet, for suturing, is a rigid arcuately-shaped needle for use during a suturing operation.

It is still further desirable to grasp a needle with an instrument that would be deliverable through narrow, convoluted working channel of a flexible endoscope, and yet would be capable of grasping the needle firmly and securely.

It is desirable to provide an associated method for suturing through an endoscope, supplementing or replacing the more invasive surgical procedures, and reducing the complications and drawbacks of existing open or laparoscopic surgical procedures particularly those performed for morbid obesity.

The benefits of the present invention in addressing the drawbacks and shortcomings of the prior art and the objectives and needs noted above will be more readily apparent from the description and drawings of the invention set forth herein.

### SUMMARY OF THE INVENTION

The present invention is directed to a surgical endoscopic suturing system to be used in conjunction with an endoscope. The invention relates to suturing of internal body tissues as part of a surgical procedure which may be diagnostic, therapeutic or both. In accordance with the present invention, there is provided an endoscopic surgery system comprising a temperature biased suture needle, a needle grasping device, and an elongated catheter or other delivery tube, a endoscopic surgery system configured for use in conjunction with an endoscope insertion member material that may become transformed from a malleable to a rigid state and vice versa. As such, a suture needle is sufficiently malleable to be passed through the working channel of the flexible endoscope. When a needle is ejected from the working channel in readiness for suturing, it may be treated in a particular manner to transform a needle into a rigid state, appropriate for suturing tissue.

In one embodiment of the present invention, the suture needle is configured of a temperature biased shape memory alloy Nitinol (NiTi). The Nitinol alloy selected for a needle takes on a desired arcuate shape and stiffness appropriate for suturing when heated to a certain temperature. When cooled below a specific temperature, it does, in turn assume a malleable state. The ability to return to the previously defined shape when subjected to the appropriate thermal procedure is the basis upon which the temperature biased suture needle functions in accordance with the principles of the present invention. Accordingly, the temperature at which the suture needle will be in a heated state may vary. For example, in one embodiment, the suture needle is in a heated state at a temperature proximate body temperature. In another embodiment, the suture needle is in a heated state at a temperature above body temperature.

The needle-grasping device manipulates the suture needle. Pursuant to a particular feature of the present invention, the needle-grasping device is configured to firmly grasp the suturing needle, enabling a needle's passage through the working channel of the endoscope insertion member, and performance of the suturing operation in a consistent and reliable manner. Pursuant to an embodiment of the present invention, the needle-grasping device is made of a rigid material such as stainless steel, and is comprised of a handle mechanism, a long flexible shaft, and a jaw assembly. According to a particular feature of the present invention, the jaw assembly is configured such that the inner surfaces of the grasping jaws possess a series of ridges, specially designed to firmly grasp the suture needle thereby preventing its rotation on its own axis during the suturing operation. The control mechanism for opening and closing the jaw assembly is comprised of one or more wires traversing through the shaft of a needle grasping device, a wires being configured to transmit mechanical compressive and tensile forces to enable alternating opening and closing of jaws. The wire(s) are operatively connected to a handle mechanism proximally, and to jaw assembly distally.

In one embodiment of the needle-grasping device pursuant to the present invention, the handle mechanism comprises two finger rings operatively coupled with two leverage joints, a leverage joints being operatively connected with the wire that traverses the shaft of a needle-grasping device, the distal end of a wire being coupled with the jaw assembly. When said finger rings of the handle mechanism are pulled apart, a leverage joints are co-jointly pulled in opposing directions, thereby relaxing the pull on the wire. The relaxation of the wire causes said jaws to open. When the finger rings of the handle mechanism are approximated together thereby approximating a leverage-joints, a strong pull is created and applied onto the wire, causing the jaws to close tightly, thereby enabling a firm grasp of the suture needle.

The delivery tube or tubular member is configured to house the needle grasping device and suture needle while being passed through the working channel of the endoscope insertion member. In one particular embodiment of the present invention, a collar comprises the distal end of the delivery tube, serving to protect the working channel of the endoscope insertion member from the sharp needle point, while enabling its exit from the flexible shaft of a delivery tube without piercing it. A locking mechanism may be included in the handle mechanism in order to lock said jaws in a closed position over the needle during the suturing operation.

An additional alternative embodiment of the present invention, wherein the temperature control system utilizes electricity for providing heat to the suture needle, includes an electrical source providing electrical power, such as an electrical generator. The electrical source is operatively connected to an electrical connector and current is passed through said electrical connector and through an appropriate low resistance connection that is coupled to one or both of the high resistance metal jaws of the needle-grasping device. This delivery of power (e.g., electrical current) to a jaw assembly causes the jaws, and subsequently the needle that is being grasped by said jaws to become heated, thereby transforming a needle into its austenitic state. When the suture needle requires withdrawal at the termination of the procedure, cold water may be injected through the designated channel in the needle-grasping device directed to flow over a suture needle, thus rendering it malleable for withdrawal.

Alternatively, the collar that comprises the distal end of the delivery tube may be heated to direct heat to the needle. In another embodiment of the present invention, a delivery tube includes insulated low resistance wires coupled to a connector. The wires may extend along and be imbedded in the shaft of delivery tube. An electrical source is operably connected to an electrical connector thereby passing electrical power through said electrical connector and down the low resistance wires. The wires are distally connected to a high resistance metal collar. As current is transmitted along this embodiment, the metal collar becomes hot, thus transmitting heat to the needle thereby causing it to assume its arcuate rigid state. Upon the need for withdrawal, cold water is injected as described.

An associated minimally invasive surgical suturing method utilizes the above-described endoscopic surgical suturing assembly and comprises inserting a distal end portion of the endoscope insertion member into a patient in order to visualize the targeted tissue for suturing. The method further comprises inserting the suture needle grasped by needle grasping device, a needle-grasping device being housed inside the delivery tube, into the working channel of the endoscope insertion member. Upon visualization of target tissue in need of a suturing, a tubular member-containing needle grasping device and needle is ejected from the working channel of the endoscope, while a needle is in its malleable, martensitic state. The suture needle is then positioned proximate the target tissue, and heated by utilizing the temperature control system preferably by injecting hot water, thereby transforming a needle to its arcuate, stiffened austenitic state in preparation for the suturing operation. Upon transformation of a needle to its suturing state, the operator manipulates the needle through target tissue by means of the endoscopic insertion member and needle-grasping device, thus performing the suturing operation. Upon completion, state of the art endoscopic scissors are utilized to sever the suture thread. Thereafter, cold water is injected through the channel in the delivery tube or the needle grasping device, thereby transforming a needle to its malleable, martensitic state in preparation for withdrawal of a needle from the patient through the working channel of a endoscope insertion member.

These embodiments and others are described in further detail below.

### BRIEF DESCRIPTION OF THE DRAWINGS

A complete understanding of the present invention may be obtained by reference to the accompanying drawings, when considered in conjunction with the subsequent, detailed description.

FIG. 1 is a schematic perspective view of the distal end of the endoscopic surgical assembly in accordance with the present invention.

FIG. 2 is a schematic perspective view of an embodiment of a suture needle.

FIG. 3 is a schematic perspective view depicting a temperature biased suture needle in the malleable (martensitic) state emerging from the working channel of an endoscope.

FIG. 4 is a schematic perspective view depicting an endoscope employing the suturing assembly approaching the tissue targeted to be sutured.

FIG. 5 is a schematic perspective view depicting a curved suture needle in its stiff (austenitic) state being held by the needle-grasping device just prior to introduction into the targeted tissue.

FIG. 6 is a schematic perspective view depicting the curved suture needle in its stiff (austenitic) state being maneuvered by the endoscope to enter the targeted tissue.

FIG. 7 is a schematic perspective view depicting the curved suture needle in its stiff (austenitic) state emerging from the tissue and being re-grasped by the needle-grasping device.

FIG. 8 is a schematic perspective view of the suture needle in its malleable (martensitic) phase being pulled back into the working channel of the endoscope after completing one stitch of the suturing operation.

FIG. 9 is a schematic perspective view of a suture needle depicting the needle shaft configured in a triangular “cutting” shape, and the needle's proximal end shaped with ridges.

FIG. 9A is a schematic perspective end view depicting a needle-grasping device holding a needle.

FIG. 9B is a schematic perspective end view depicting yet another needle grasping device holding a needle.

FIG. 9C is a further schematic perspective end view depicting a needle-grasping device holding a needle.

FIG. 10 is a schematic perspective view of the distal end of the delivery tube and needle-grasping device, depicting a fluid port built into the wall of the delivery tube.

FIG. 11 is a schematic perspective view of one embodiment of the present invention assembly depicting the proximal end of a needle-grasping device with an electrical temperature control system.

FIG. 12 is a schematic perspective view of one embodiment of the present invention assembly depicting perspective view of the needle grasping device configured injection of fluid onto the temperature biased suture needle.

FIG. 13 is a schematic perspective view of the needle grasping device jaw assembly configured with a ridged surface disposed on the inner aspect of each jaw.

FIG. 13A is a further schematic perspective view of the needle grasping device jaw assembly configured with a ridged surface disposed on the inner aspect of each jaw.

FIG. 14 is a schematic perspective view of the present invention depicting a fluid channel configured into the shaft of a needle-grasping device.

FIG. 15 is a schematic perspective view of another embodiment of the invention depicting the needle-grasping device coupled to an electrical source.

FIG. 16 is a schematic perspective view of yet another embodiment of the invention depicting the needle-grasping device with low electrical resistance conductive wires imbedded along its shaft.

FIG. 17 is a schematic view of an embodiment of the needle grasping device handle assembly in the open configuration in accordance with the present invention.

FIG. 18 is a schematic view of an embodiment of the needle grasping device jaw assembly in the open configuration in accordance with the present invention.

FIG. 19 is a schematic view of an embodiment of the needle grasping device handle assembly in the closed configuration in accordance with the present invention.

FIG. 20 is a schematic view of an embodiment of the needle grasping device jaw assembly in the closed configuration in accordance with the present invention.

FIG. 21 is a schematic side view of an embodiment of a grasping jaw.

FIG. 22 is a perspective view of the jaw in FIG. 21.

FIG. 23 is a sectional side view of the jaw in FIG. 21.

For purposes of clarity and brevity, like elements and components will bear the same designations and numbering throughout the figures.

### DESCRIPTION OF THE PREFERRED EMBODIMENTS

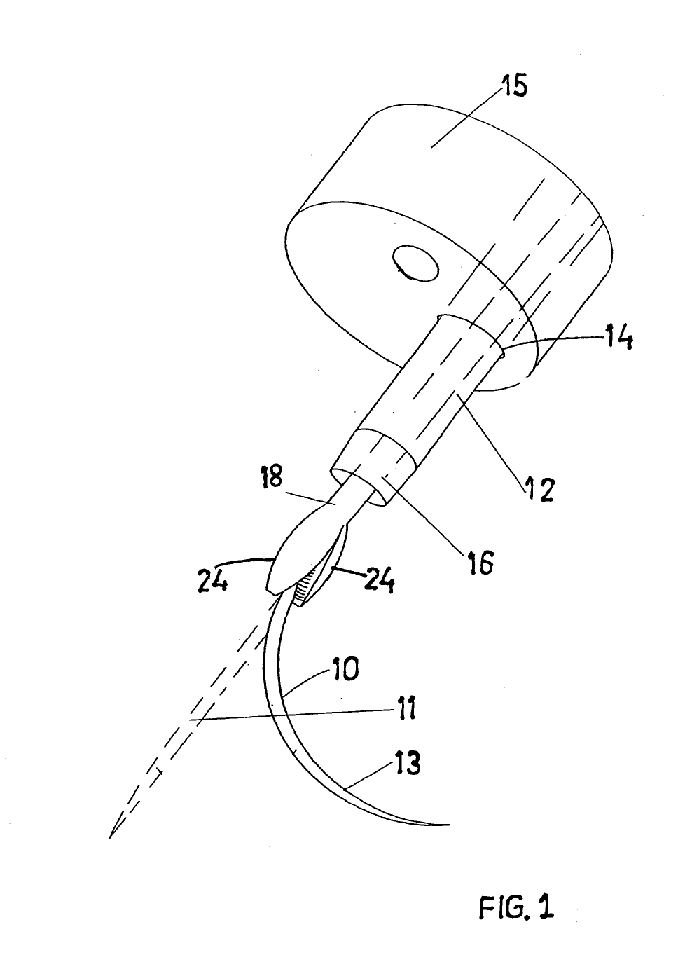

As illustrated in FIG. 1 an endoscopic surgery system or surgical assembly comprises a temperature biased suture needle 10, a needle-grasping device 18, and an elongated catheter or other delivery tube or tubular member 12, shown emerging from working channel 14 of an endoscope insertion member 15. Delivery tube or tubular member 12 is configured for insertion into the working channel 14 of endoscope insertion member 15. Needle grasping device 18 includes a flexible or rigid elongated shaft, a handle mechanism and a jaw assembly with jaws, and is movable within delivery tube 12 and is configured for grasping and manipulating suture needle 10. Suture needle 10 is shown in a straight malleable (martensitic) state 11 in dashed lines, and in a stiff, hardened, austenitic state 13. Suture needle 10 is in its malleable state 11 for passage through or manipulation inside working channel 14 of endoscope insertion member 15, and in its hardened arcuate shape for suturing tissue. Suture needle 10 illustrated in FIG. 1 is a temperature biased suture needle, whereby in one particular embodiment it is made of a nickel titanium (NiTi) alloy such as Nitinol. In the embodiment of a needle in accordance with the present invention, the Nitinol alloy selected for the needle 10 takes on a desired shape (arcuate) and stiffness appropriate for suturing when heated to a certain temperature, and becomes malleable when cooled below a specific temperature.

The term Shape Memory Alloys (SMA) is applied to that group of metallic materials that demonstrate the ability to return to some previously defined shape or size when subjected to a certain strain. Although a relatively wide variety of alloys are know to exhibit the shape memory effect, it is preferable, in accordance with the principles of the present invention to use a specific shape memory alloy that can return to a previously defined shape when subjected to the appropriate thermal procedure. This material can be plastically deformed at some relatively low temperature, while upon exposure to a higher temperature will return to its predetermined shape prior to the deformation. When such a temperature biased SMA is subjected to temperatures below its transformation temperature, it has very low yield strength and can be deformed quite easily into any new shape. However, when the material is heated above its transformation temperature it undergoes a change in crystal structure that causes it to return to its original shape.

The mechanical properties of temperature biased SMAs vary greatly over the temperature range spanning their transformation. The martensite (malleable low temperature phase) is easily deformed to several percent strain at quite a low stress, whereas the austenite (stiff high temperature phase) has much higher yield and flow stresses. Upon heating, the metal remembers its unstrained shape and reverts to it as the material transformed to austenite.

The basis of the nickel-titanium system of alloy is the binary, equiatomic intermetallic compound of NiTi. The intermetallic compound is extraordinary because it has a moderate solubility range for excess nickel or titanium, as well as most other metallic elements, and it also exhibits ductility comparable to most ordinary alloys. This solubility allows alloying with many of the elements to modify the temperature transformation properties of the system. Excess nickel, in amounts up to about 1%, is the most common alloying addition. Excess nickel strongly depresses the transformation temperature and increases the yield strength.

In accordance with the present invention, the needle of the invention is made of a shape memory alloy of nickel and titanium. The needle has a special ratio of Ni to Ti, whereby it assumes a malleable, or martensitic, state when chilled and a rigid, or austenitic, state when heated. In one embodiment, the Ni to Ti ratio is such that the transition temperature from martensitic (malleable) to austenitic (rigid) is between 30° C. (±3) and 39° C. (±3°). In a more specific embodiment, the Ni to Ti ratio is such that the austenite (rigid) start temperature (Aₛ) is in the range of 30° C. and its austenite (rigid) finish temperature (A_{f}) is in the range of 39° C. In another embodiment, the needle assumes its austenitic (rigid) state at a temperature proximate body temperature. In still another embodiment, the needle assumes its austenitic (rigid) state at a temperature above body temperature.

Delivery tube 12 is shown in FIG. 1 with metal collar 16 at its distal end. Collar 16 is configured to protect the suture needle tip during insertion of the suturing assembly through the endoscope working channel. Metal collar 16 may also be used to transmit heat to suture needle 10 thereby activating the austenitic arcuate (curved) form of a suture needle 10. As discussed in detail below, collar 16 may be coupled to a temperature control system that may include a standard electrosurgical generator. When a generator is coupled with the suturing assembly and activated, an electrical current would be transmitted to collar 16 heating it, and thereby transmitting a heat to suture needle 10. Delivery tube 12 might also contain one or more hollow lumens or channels, at least part way along the wall of the tubular member and configured for directing fluid from a port located in or near the proximal handle assembly onto to suture needle 10. Needle grasping device 18 is used to guide suture needle 10 out of delivery tube 12 and may preferably also be used as a source of heat for suture needle 10 to activate the shape memory. As such, needle grasping device 18 may be coupled to the electric temperature control system and/or have one or more hollow lumens or channels longitudinally along its shaft or in its shaft proximally coupled to a port in or near the handle assembly of needle grasping device 18 and used as discussed further herein.

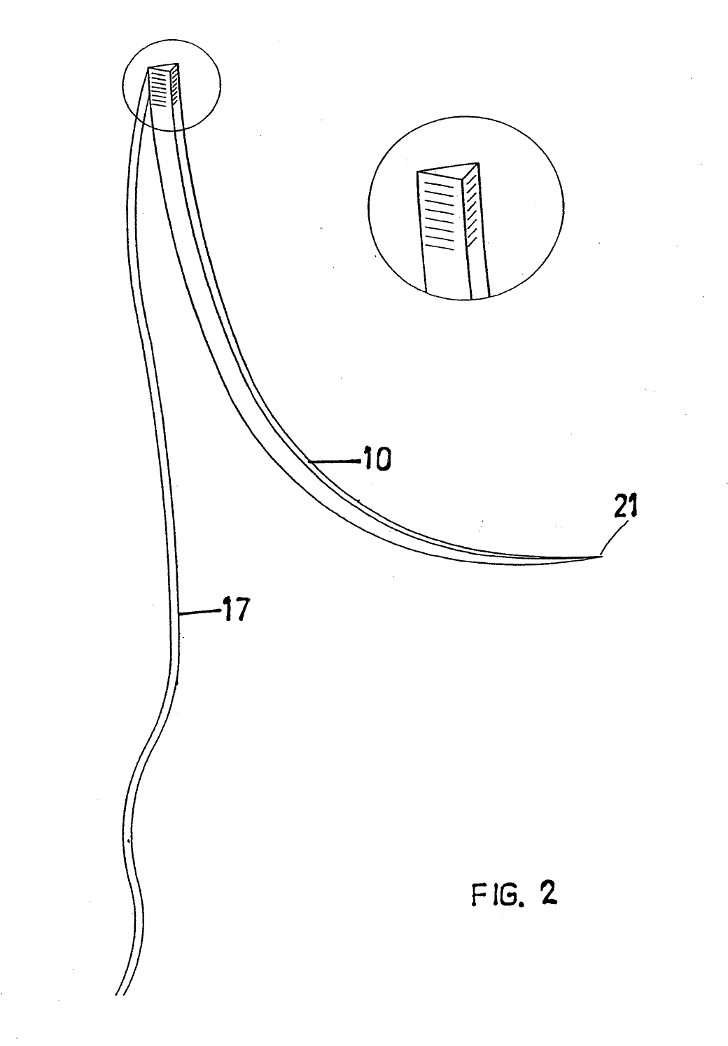

FIG. 2 is a schematic perspective representation of suture needle 10 in its curved austenitic state, coupled with a suitable suture 17. Suture needle 10, in one embodiment, is made from a temperature biased shape memory metal, for example, Nitinol, and is configured to include a sharp distal tip 21 for piercing tissue. In this preferred embodiment of the present invention, Needle 10 is depicted to have a shaped shaft with a generally triangularly shaped cross-section with sharpened cutting angles for easy passage through tissue. The proximal end 20 of suture needle 10 is securely attached to an appropriate length of suture thread 17, such as biocompatible glue, for example.

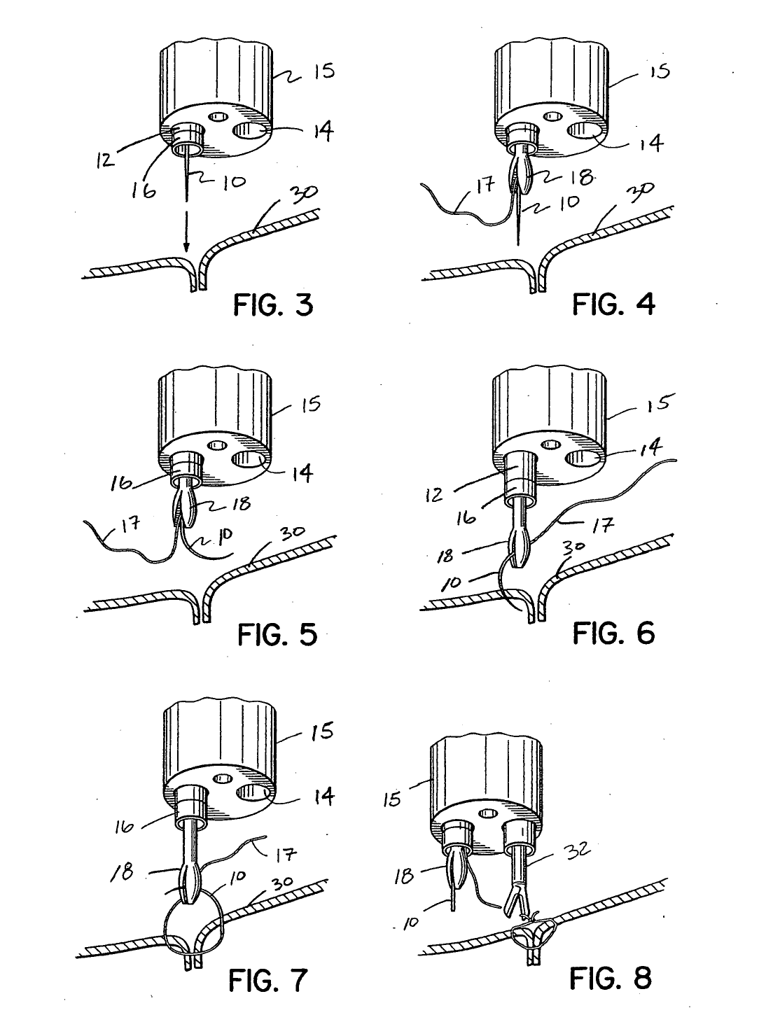

FIGS. 3-8 are schematic views of an endoscope employing the endoscopic surgical assembly of FIG. 1, showing successive steps in a suturing operation pursuant to the invention. FIG. 3 is a schematic perspective view of suture needle 10, delivery tube 12 with metal collar 16 emerging from the working channel 14 of a multi-channel endoscope insertion member 15. Targeted tissue 30 to be sutured is shown as well. Suture needle 10 is in a generally straight configuration and is in its malleable martensitic phase at a temperature below the heated state as it is ejected from the distal end of the delivery tube 12 by needle grasping device 18 housed inside delivery tube 12.

FIG. 4 is a perspective view of suture needle 10, needle grasping device 18 and metal collar 16 emerging from one working channel of endoscope insertion member 15. In this figure needle-grasping device 18 is holding suture needle 10 coupled with suture 17. The needle is still in the malleable martensitic state because heat has not yet been applied to suture needle 10 to activate its shape memory.

FIG. 5 is a schematic perspective view of needle grasping device 18 holding curved suture needle 10. Needle 10 is in its rigid, curved, austenitic state after heat has been applied to it in order to activate its shape memory. Suture 17 and targeted tissue 30 to be sutured are shown as well.

FIG. 6 is a schematic perspective view of needle grasping device 18 emerging from delivery tube 12 with metal collar 13, a suturing assembly being employed by endoscope insertion member 15. Needle grasping device 18 has a firm hold on curved suture needle 10, a needle being guided into target tissue 30 with the aid of endoscope insertion member 15, and needle-grasping device 18. Suture 17 is securely attached to proximal end of suture needle 10.

FIG. 7 is a schematic perspective view of curved suture needle 10 emerging from targeted tissue 30 and being captured by needle grasping device 18. Suture 17 is pulled through targeted tissue 30 as suture needle 10 is passed, thus forming a loop of suture 17, which may be tied to approximate and secure tissue in the desired position.

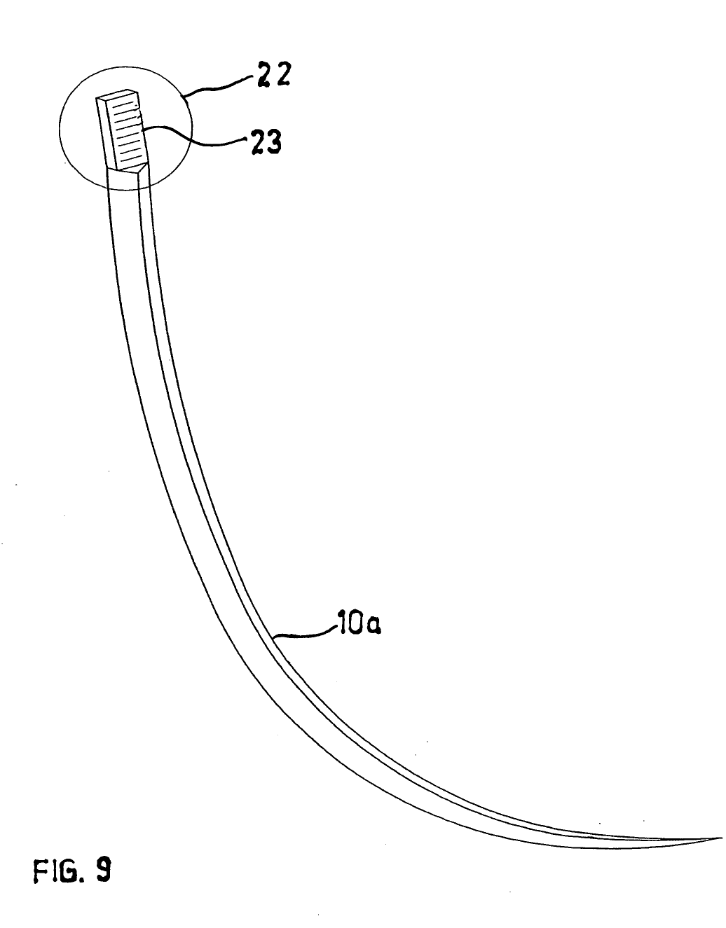

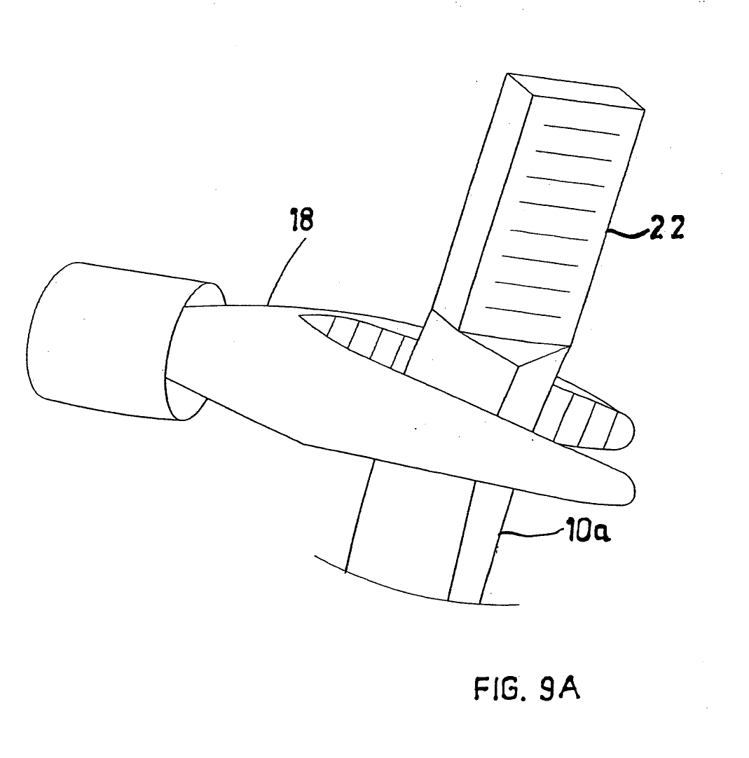

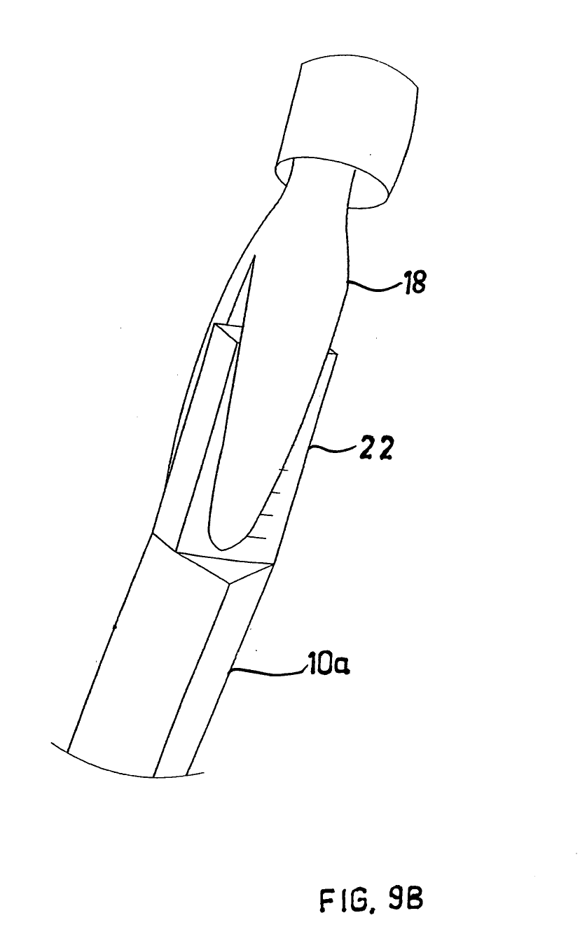

FIG. 8 is a schematic perspective view of the distal end of suture needle 10 being pulled back into delivery tube 12 while a suture line cutter 32 is passed through another working channel 14 of the endoscope insertion member 15. Suture cutter or scissors 32 is used to sever suture needle 10 from suture 17 after a knot has been tied to secure tissue in preferred position. Upon grasping of suture needle's distal tip by needle grasping device 18, cooling of suture needle 10 may take place by injection of cold water through a specially allocated channel in needle grasping device 18, or delivery tube 12, to be shown further below. This cooling process transforms suture needle 10 into its malleable or martensitic state, thus facilitating its removal through the working channel 14 of endoscope insertion member 15, along with excess suture 17.

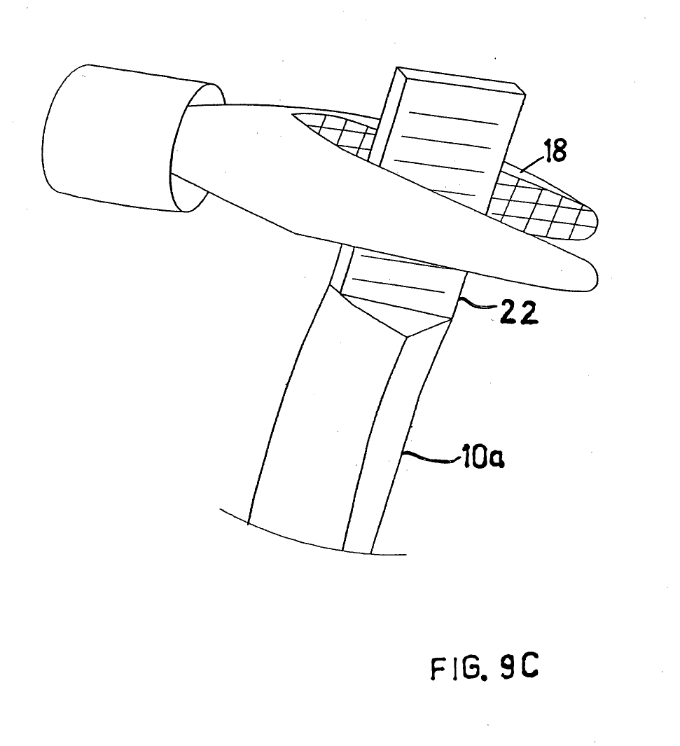

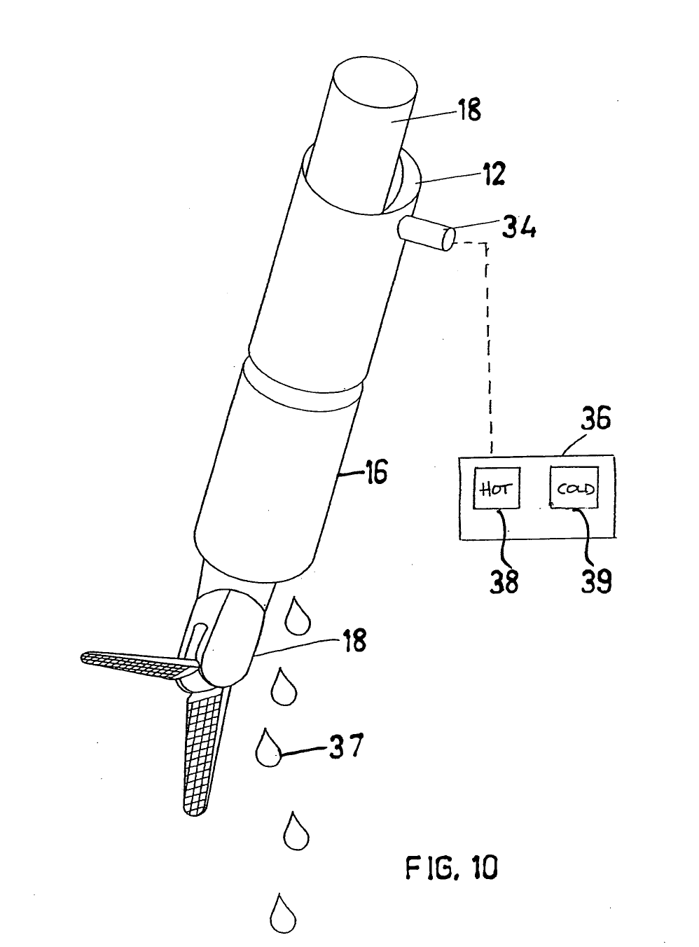

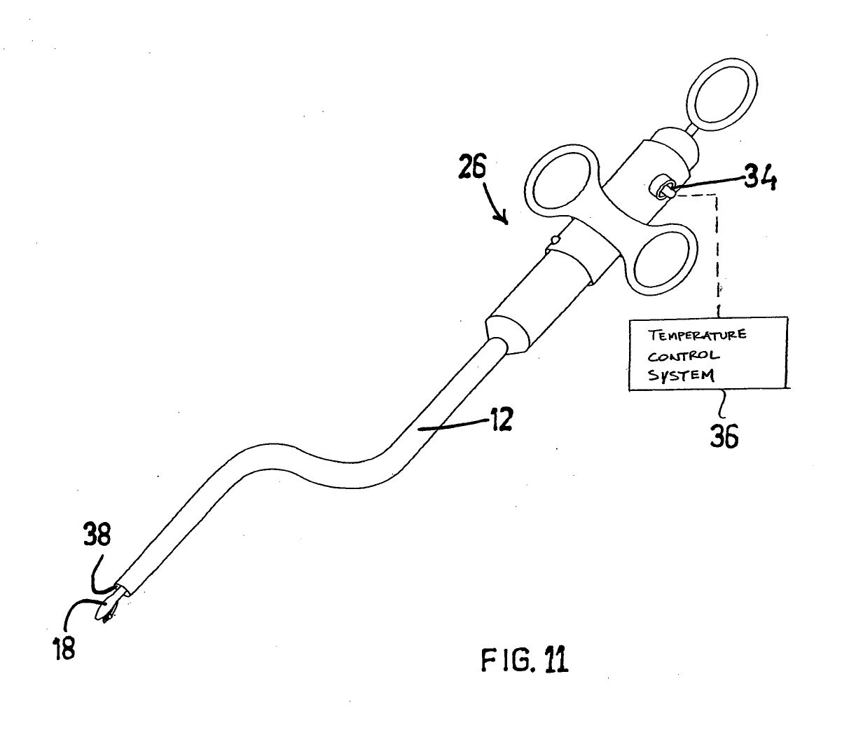

FIG. 9 is a schematic perspective view of an alternative embodiment of suture needle 10 wherein the shaft of suture needle 10a has a cross-section with a generally triangular shape, with the three angles of the triangle being sharply formed, configured for cutting and easy passage through the tissue. The proximal end 22 of needle 10a is flattened with a rectangular cross section, a flattened portion's surfaces configured with a series of ridges 23, a ridges corresponding to similar ridges located in the inner surface of the jaw assembly of needle grasping device 18, (FIG. 10) to provide a better hold of the needle by a jaws. Most particularly, the hold that is desired is one that would not allow for suture needle 10a to rotate on its own axis during the process of suturing tissue.

FIGS. 9A, 9B, and 9C illustrate alternative means and embodiments for grasping and manipulating needle 10a with needle grasping device 18. Alternatively, the proximal end of suturing needle 1 may be constructed in a triangular, rectangular or circular cross section. The cross-sectional configuration of a portion of the needle may be circular, rectangular, or triangular.

FIG. 10 represents a perspective view of one embodiment of the present invention wherein heated or cooled fluid 37 is used to transform suturing needle 10 from an austenitic to a martensitic state, and vice versa. A fluid port 34 is coupled to one or more channels disposed or fashioned longitudinally along the delivery tube 12 for directing warm fluid through a channel in tube 12 and out its distal end for the purpose of bathing a needle and transforming it into its hardened arcuate state. Delivery tube 12 might include a separate channel 35 for the fluid 37 or the fluid may traverse through the passage or channel, which extends through the delivery tube 12 in which the needle-grasping device 18 is positioned. When suture needle 10 requires withdrawal through working channel 14 of endoscope insertion member 15, fluid port 34 is utilized in order to direct cooled or cold fluid to bathe suture needle 10, thereby transforming it into its malleable state. Fluid port 34 may be coupled with a temperature control system 36 that includes supplies of hot 38 and cold 39 fluids, or fluids of varying temperature. A syringe, for example may be used for the purpose of injecting fluid to port 34.

FIG. 11 represents a schematic perspective view of an alternative embodiment showing the port 34 located proximate the handle 26. Port 34 is operably coupled with delivery tube 12 and with a temperature control system that may include heated or cooled fluid (FIG. 10). Warm fluid may be injected into injection port 34 and directed along delivery tube 12 to exit at fluid port 38 at the distal end of the tube 12 bathing the suture needle and thereby causing its transformation into the hardened state. Alternatively, when the needle requires withdrawal, cold water is injected rendering suture needle 10 malleable.

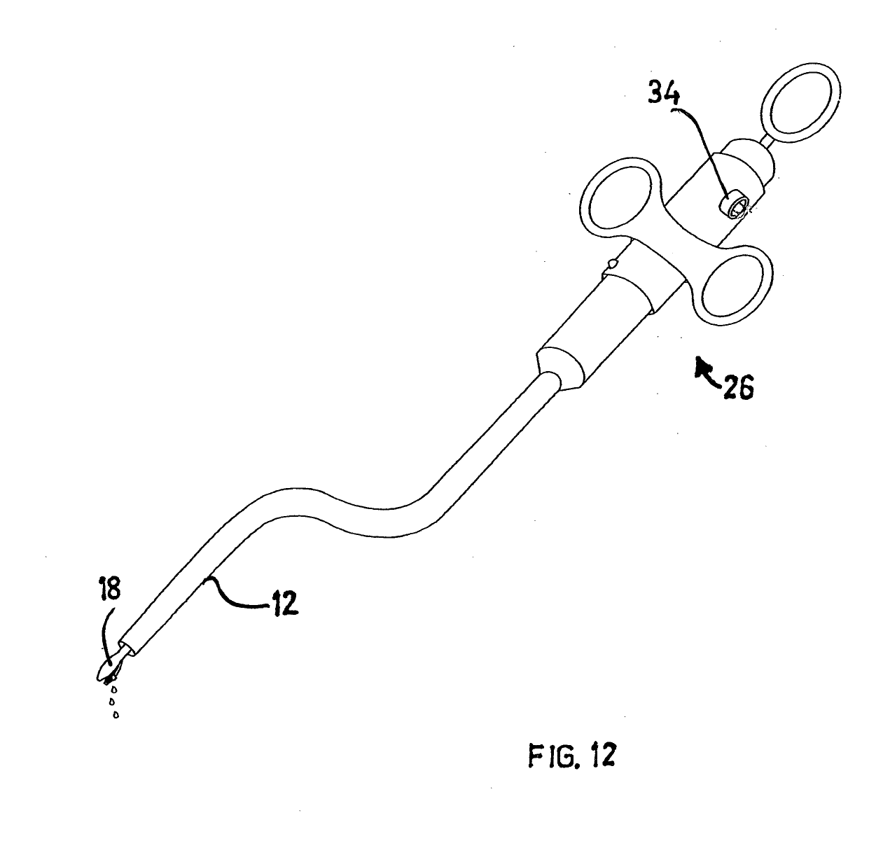

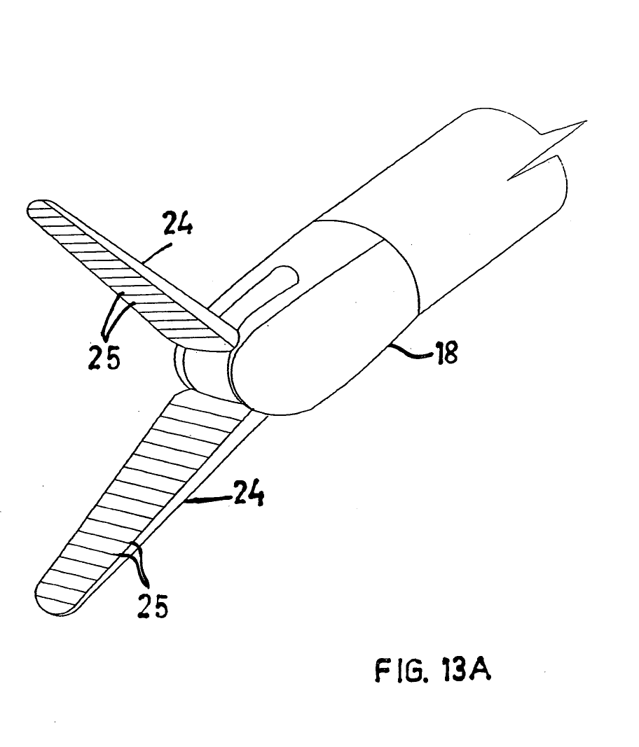

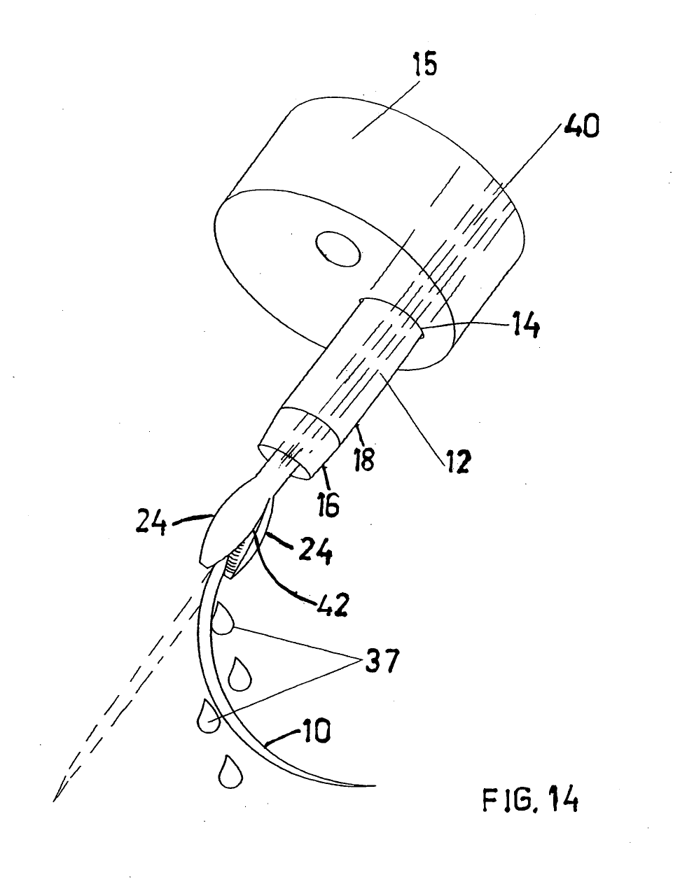

FIGS. 12 and 14 illustrate a schematic perspective representation of another embodiment of the invention wherein the fluid is directed through the needle-grasping device. Referring to FIG. 14, the needle-grasping device includes a fluid channel 40, which extends along at least a portion of the length of the needle-grasping device and is coupled with injection port 34 (FIG. 12). The fluid channel 40 terminates in an outlet 42 proximate jaw assembly 24 of the needle holding device. In this preferred embodiment, fluid channel 40 conducts heated or cooled fluid 37, injected into injection port 34, to fluid outlet 42 at the distal end of needle grasping device 18, causing the desired deformation and shaping of suture needle 10, in accordance with the invention.

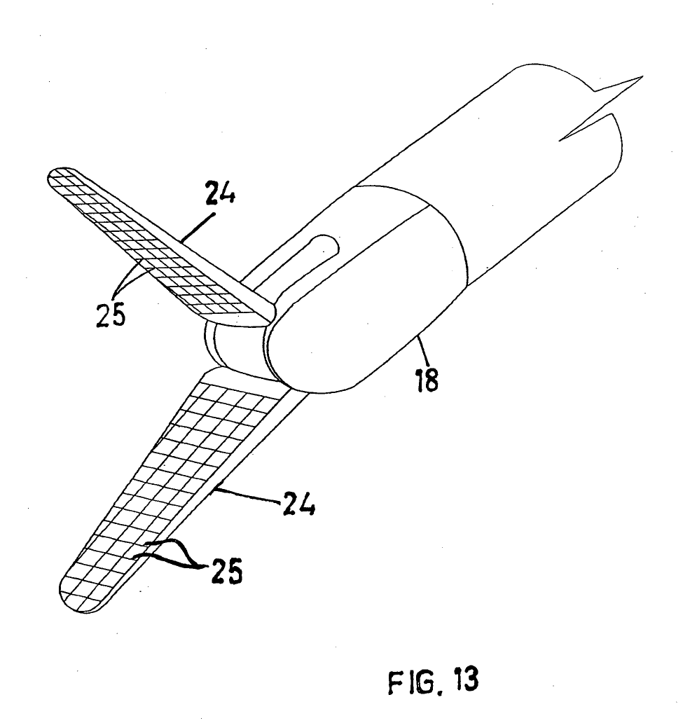

FIGS. 13 and 13A are perspective views of needle grasping device jaws 24 with ridges 25 placed onto the inner surfaces of said jaws. Ridges may be cut into various patterns with embodiments having vertical or horizontal ridges down the inside of both jaws (FIG. 13A). Another embodiment may be configured with diagonal ridges, while another, with ridges cut into a checkerboard pattern (FIG. 13) or diamond pattern.

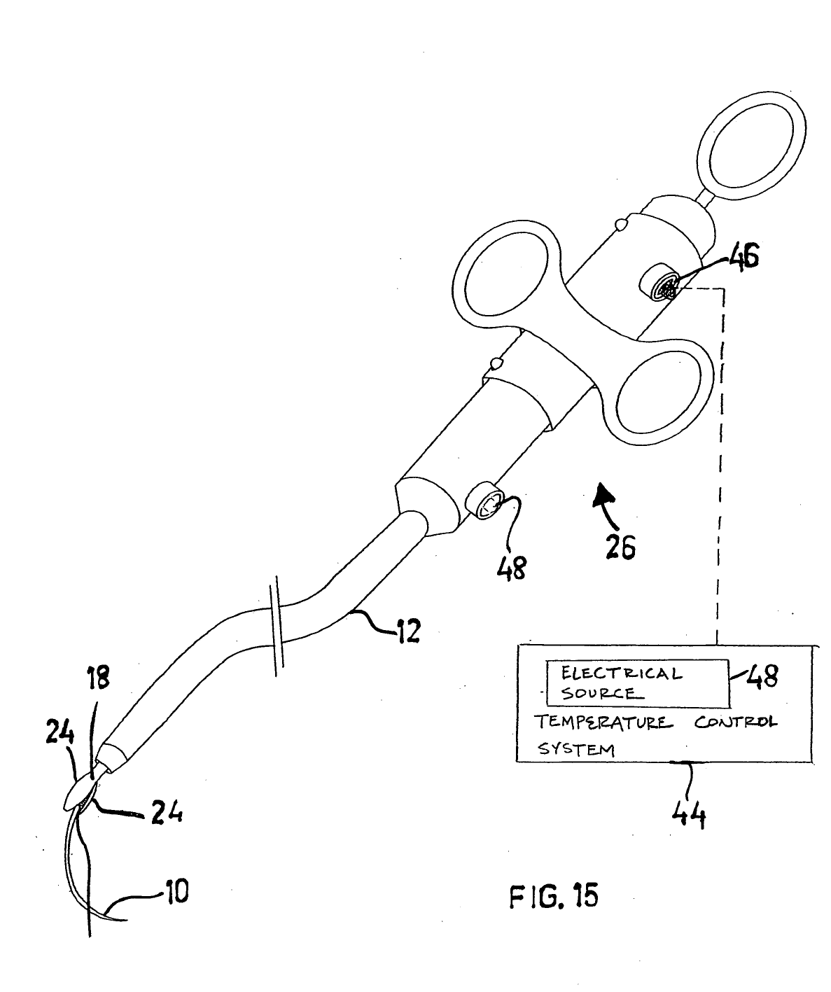

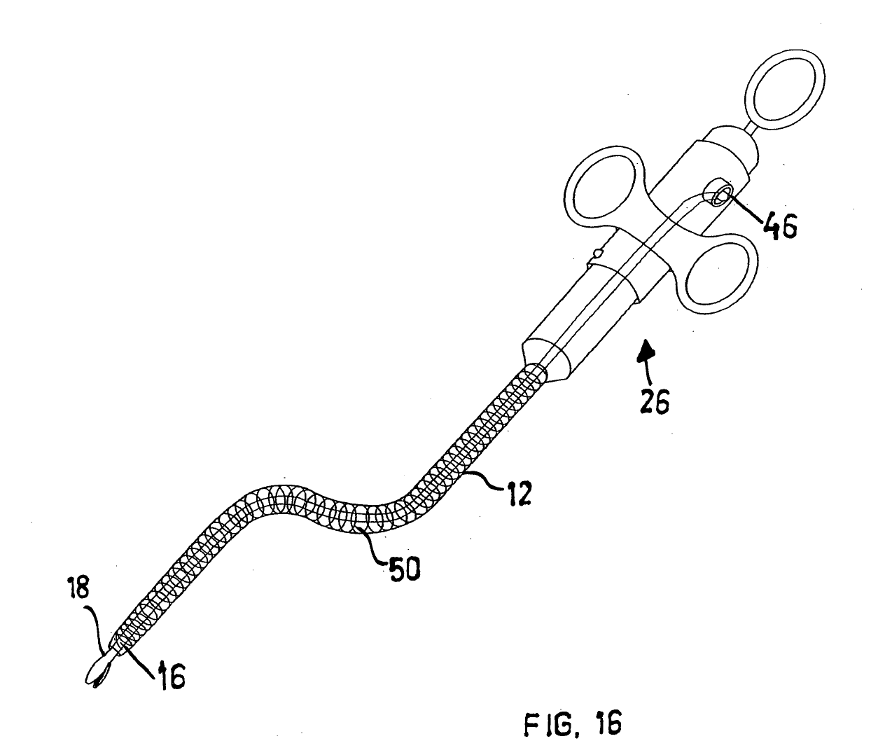

FIGS. 15 and 16 illustrate additional alternative embodiments of the invention wherein the temperature control system is electric in nature and utilizes electricity for providing heat to the suture needle 10. The embodiment depicted in FIG. 15 illustrates a temperature control system 44 that includes an electrical source 48 providing electrical power, such as an electrical generator. Electrical source 48 is operatively connected to an electrical connector 46 and electric current is passed through electrical connector 46 and through an appropriate low resistance connection that is coupled to one or both of the high resistance metal jaws of the needle grasping device 18. This delivery of power (e.g., electrical current) to jaw assembly 24 causes the jaws, and subsequently the needle that is being grasped by said jaws to become heated, thereby transforming needle 10 into its rigid state. When suture needle 10 requires withdrawal at the termination of the procedure, cold water may be directed toward the distal end of delivery tube 12 flowing over suture needle 10 rendering suture needle 10 malleable for withdrawal as discussed above. A locking mechanism may be included in the handle mechanism in order to lock jaws 24 in a closed position over the needle during the suturing operation. The system might include a lock button 48 for this purpose (FIG. 15). Alternatively, collar 16 of delivery tube 12 may be heated to direct heat to the needle. FIG. 16 illustrates an embodiment that includes insulated low resistance wires 50 coupled to connector 46. The wires may extend along and be imbedded in the shaft of tubular number or delivery tube 12. An electrical source 48 may be operably connected to electrical connector 46 thereby passing electrical power through electrical connector 46 and down the low resistance wires 50. The wires 50 are distally connected to high resistance metal collar 16. As current is transmitted along this embodiment, metal collar 16 becomes hot, transmitting heat to needle 10, and causing it to assume its arcuate rigid state. Upon the need for withdrawal, cold water is injected as described above.

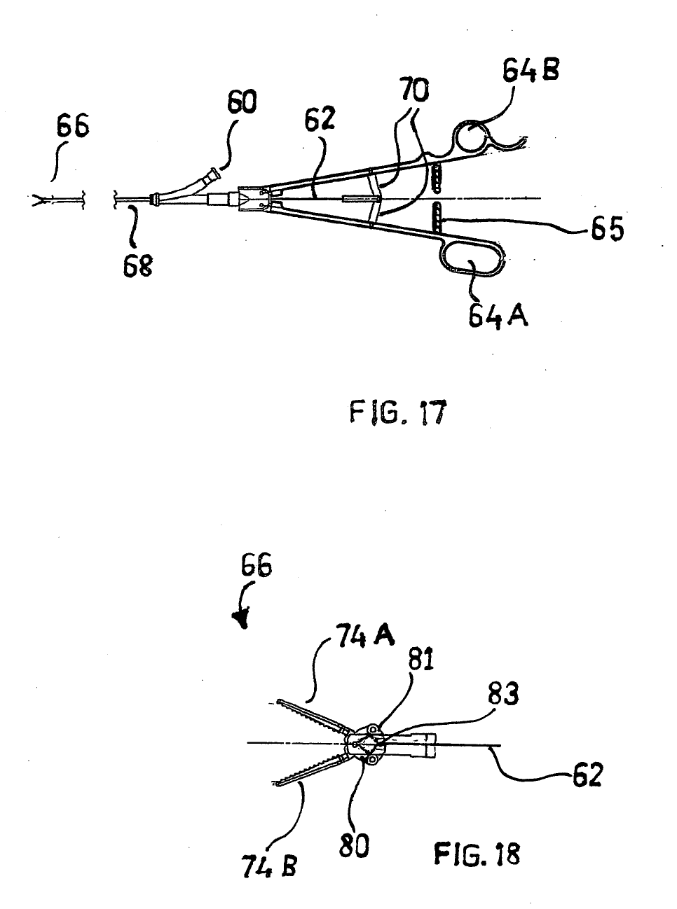

FIG. 17 illustrates another embodiment of the needle-grasping device in an open configuration. Push-pull wire 62 traverses through shaft 68 or is incorporated into the shaft and is operably connected to jaw mechanism 66 distally and leverage-joints 70 proximally. When opposing scissor finger rings or handles 64A and 64B of the device are pulled apart or separated, leverage-joints 70 are co-jointly pulled in opposing directions or separated, thereby relaxing the pull on wire 62 and moving the wire in a distal direction toward jaws 74A, 74B causing jaws 74A and 74B to open. FIG. 18 is a detailed illustration of jaw mechanism 66 depicting jaws 74A and 74B in an open position. Flush port 60 (FIG. 17) is designed for introduction of hot or cold water, which flows through a separate channel in shaft 68 (Illustrated in FIG. 20) and bathes the temperature biased suture needle in jaw mechanism 66. The needle-grasping device includes a ratchet locking structure 65 to hold the scissor finger rings together tightly to grasp the needle.

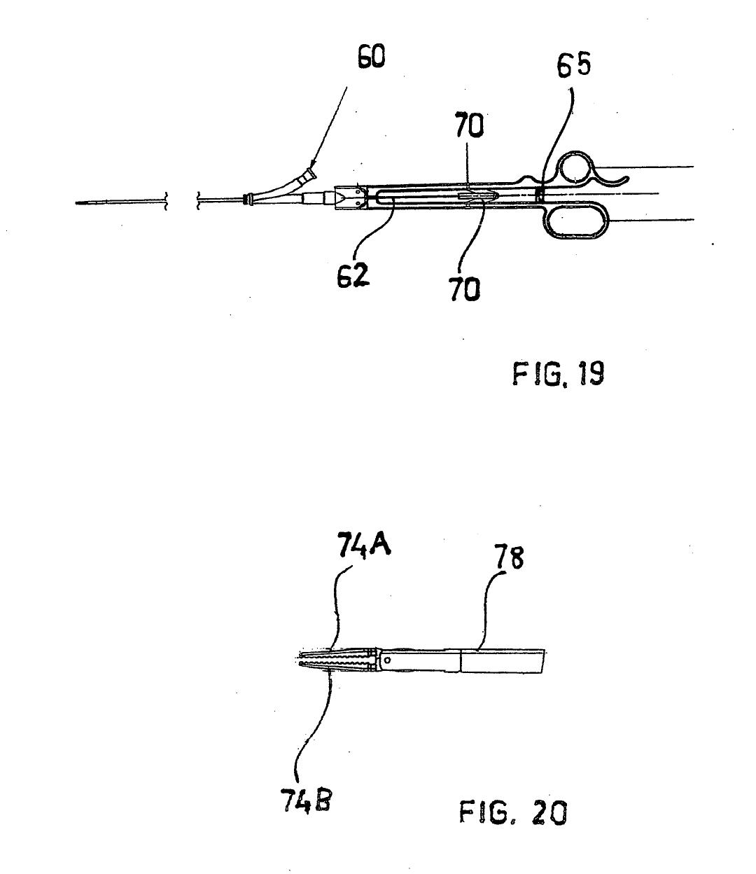

FIG. 19 illustrates the preferred embodiment of the needle grasper in a closed configuration. Scissor finger rings 64A, 64B are approximated, causing leverage-joints 70 to be brought together or approximated, thereby applying a strong pull on wire 62. As a result, jaws 74A and 74B are approximated together tightly, allowing for a firm grasp of the suture needle. The ratchet locking structure 65 is shown locked to hold the jaws together. FIG. 20 is a detailed schematic representation illustrating tightly closed jaws 74A and 74B. Flush channel 78 that communicates with flush-port 60 is illustrated in FIG. 20.

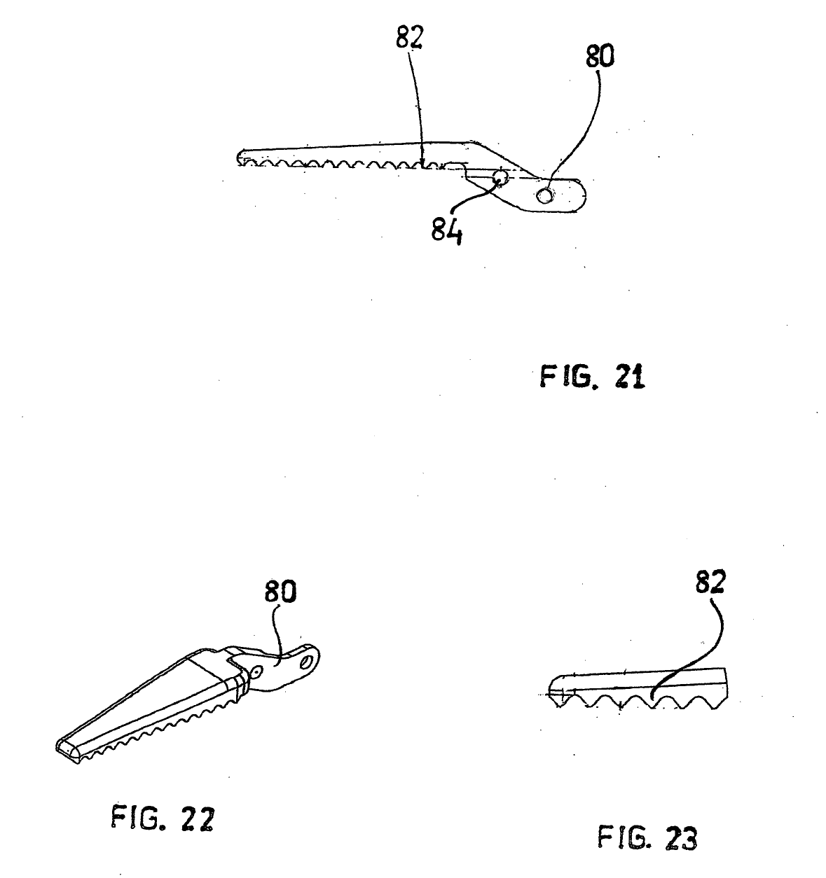

FIGS. 21 and 22 depict a detailed drawing of jaws 74A and 74B. The proximal aspect of the jaws, namely leg 80, is operatively coupled to wire 62, allowing for secure closure of the jaws. The jaws 74A, 74B each include a leg 80 and a pivot opening 84 to receive a pin or other pivot element for pivoting. The legs 80 are at an angle to the toothed portion of the jaws. As the legs are spread apart, the jaws spread apart (FIG. 18). Scissor linkages 81 are pivotally coupled to each leg 80 at respective pivot points or pivot pins at one end. The other ends of the scissor linkages 81 are appropriately coupled at another pivot point 83 to cable 62. When scissor ring fingers 64A, 64B are brought together, the cable 62 slides in a distal direction and cable 62 is pushed or relaxed (FIG. 17). The distance between the pivot points 83, 84 is reduced and the jaws open and when cable 62 is pulled or tensioned (FIG. 19), the jaws close. FIG. 22 depicts one construction of the jaws. Their broader proximal and narrower distal ends adds leverage to the grasping force of the needle shaft. Teeth 82 situated on the inner aspect of the jaws, depicted in even greater detail in FIG. 23 are constructed so as to correspond with the ridges on the needle's proximal end, thereby providing for a secure grip of a needle.

While the present invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of applicant's general inventive concept.

1. An endoscopic surgical assembly comprising:
a tubular member;
an elongate needle-grasping device slidably disposed at least partially within a tubular member;
a suture needle with at least a portion of a needle being made of a temperature biased shape memory alloy;
and a temperature control system operable for selectively heating or cooling a needle;
wherein a temperature-biased needle may be selectively transformed between a malleable and a rigid state for use during surgery with an endoscope.

2. The endoscopic surgical assembly of claim 1 wherein a tubular member is configured for insertion into the working channel of an endoscope.

3. The endoscopic surgical assembly of claim 2 wherein a tubular member includes one or more longitudinally disposed channels extending at least partway along the wall of a tubular member.

4. The endoscopic surgical assembly of claim 3 wherein a proximal inlet of a channel is operatively coupled with an injection port, for injecting fluid.

5. The endoscopic surgical assembly of claim 4 wherein said injection port is configured for coupling with an injection syringe.

6. The endoscopic surgical assembly of claim 3, wherein a distal outlet of a channel is proximate a distal end of the tubular member.

7. The endoscopic surgical assembly claim 2 wherein the distal end of the tubular member comprises a metal ring.

8. The endoscopic surgical assembly of claim 1 wherein the elongate needle-grasping device includes a channel configured for directing fluid to the needle.

9. The endoscopic surgical assembly of claim 1 wherein a temperature control system is an electric system.

10. The endoscopic surgical assembly of claim 9 wherein the temperature control system is coupled with the needle-grasping device.

11. The endoscopic surgical assembly in claim 9 wherein the temperature control system is coupled with the tubular member.

12. The endoscopic surgical assembly of claim 11 wherein a tubular member includes a metal collar positioned at a distal end thereof, the collar being operatively coupled with a temperature control system.

13. The endoscopic surgical assembly of claim 12 wherein the temperature control system is comprised of at least one wire extending longitudinally at least partially along a wall of the tubular member and operably coupled with the metal collar.

14. The endoscopic surgical assembly of claim 1 wherein a needle-grasping device includes a flexible or rigid elongated shaft, a handle mechanism, and a jaw assembly with jaws.

15. The needle endoscopic surgical assembly of claim 14 wherein a jaw of the jaw assembly is configured with a ridged internally facing surface fashioned for grasping a suture needle.

16. The endoscopic surgical assembly of claim 14 wherein a elongated shaft includes one or more push-pull wires, the wires at least one of extending longitudinally through the shaft, or being incorporated within a shaft.

17. The endoscopic surgical assembly of claim 14 wherein a push-pull wire is operatively connected with the jaw assembly distally, and with the handle mechanism proximally.

18. The endoscopic surgical assembly of claim 14 wherein the needle grasping device includes a handle assembly, a handle assembly having opposing scissor finger rings, operatively coupled with corresponding leverage-joints that are, in turn, operatively coupled with at least one push pull wire, a push-pull wire being operatively coupled with jaws of a jaw assembly.

19. The endoscopic surgical assembly of claim 18 wherein separation of the opposing scissor finger rings causes separation of a leverage-joints and relaxation of the push-pull wire, causing a jaws to open.

20. The endoscopic surgical assembly of claim 19 wherein the approximation of the opposing scissor finger rings causes approximation of a leverage-joints, and applies a strong pull on the push-pull wire, bringing about tight closure of the jaws.

21. The endoscopic surgical assembly of claim 14, whereby the jaws are constructed with broader proximal and narrower distal ends.

22. The endoscopic surgical assembly of claim 14, whereby an inner surface of the jaws is configured with at least one of teeth or ridges.

23. An endoscopic suture needle, wherein a needle is comprised, at least partially, of a temperature biased shape memory alloy.

24. The suture needle of claim 23 wherein the needle is comprised of a shape memory alloy of nickel and titanium.

25. The suture needle of claim 23 wherein the needle is comprised of a special ratio of Ni to Ti whereby a needle assumes a malleable state when chilled, and a rigid state when heated.

26. The suture needle of claim 23 wherein a Ni to Ti ratio is such that the transition temperature from malleable to rigid is between 30° C. (±3°) and 39° C. (±3°).

27. The suture needle of claim 23 wherein a Ni to Ti ratio is such that the rigid start temperature (Aₛ) is in the range of 30° C. and its rigid finish temperature (A_{f}) is in the range of 39° C.

28. The suture of claim 23 wherein the cross sectional configuration of a portion of the suture needle is circular.

29. The suture needle of claim 23 wherein the cross sectional configuration of a portion of the suture needle is rectangular.

30. The suture needle of claim 25 wherein the needle assumes its rigid state at a temperature proximate body temperature.

31. The suture needle suture of claim 25 wherein the needle assumes its rigid state at a temperature above body temperature.

32. The suture needle of claim 23 wherein said needle is coupled at its proximal end with a suture thread.

33. The suture needle of claim 24 wherein the needle includes cavity placed into a proximal end of the needle, and the needle is coupled with a biocompatible glue.

34. A minimally invasive endoscopic suturing assembly comprising:
a tubular member;
an elongate needle-grasping device slidably disposed at least partially within a tubular member;
a suturing needle wherein at least a portion of a needle is made of a temperature biased shape memory alloy; and
a temperature control system operable for selectively heating and cooling a needle.

35. A minimally invasive surgical method for suturing comprising:
(a) providing a medical treatment assembly including:
an endoscope insertion member;
a tubular member;
an elongate needle-grasping device slidably disposed at least partially within a tubular member;
a suturing needle wherein at least a portion of a needle is configured of a temperature biased shape memory alloy; and
a temperature control system operable for selectively heating and cooling a needle;
(b) inserting a distal end portion of a endoscope insertion member into a patient;
(c) inserting a tubular member in the endoscopic insertion member with the needle being grasped by the needle grasping device;
(d) after visualizing target tissue in need of a suturing operation, ejecting said needle grasping device and needle, with the needle in a malleable state;
(e) positioning a suture needle proximate the target tissue;
(f) selectively heating a suture needle by utilizing a temperature control system, thereby transforming a needle to an arcuate, rigid state;
(g) manipulating a needle through target tissue with the needle grasping device to perform a suturing operation;
(h) applying cold liquid to the needle, thereby transforming said needle to a malleable state in preparation for withdrawal of a needle through the endoscope insertion member.

36. The surgical method of claim 35 wherein selective heating of the suture needle occurs via electricity conducted through a needle grasping device.

37. The surgical method of claim 36 wherein selective heating via electricity of a suture needle is performed by means of a metal collar positioned proximate the suture needle.

38. The surgical method of claim 35 wherein a grasping device is configured with a jaw assembly for grasping a suture needle.

39. The surgical method of claim 38 wherein a jaw assembly is configured to engage a proximally located shaped end of a suture needle.

40. The surgical method of claim 39 wherein a proximally located shaped end of the suture needle is one of a triangular, round or rectangular cross section.

41. The surgical method of claim 35, wherein selectively heating of a suture needle to transform it into its rigid state occurs at a temperature proximate body temperature.

42. The surgical method of claim 35 wherein selectively heating of a suture needle to transform it into its rigid state occurs at a temperature above body temperature.

43. The surgical method of claim 35 wherein the shape memory material may be transformed to its rigid state by heated fluid.

44. The surgical method of claim 35 wherein the shape memory material may be cooled below the malleable state by cooled liquid.

45. The surgical method of claim 35 wherein the needle is selectively heated by holding the needle with a heated needle-grasping device.

46. The surgical method of claim 35 wherein the needle is selectively heated by holding the needle proximate to a heated element.
